(19) United States
(12) Patent Application Publication
(10) Pub. No.: US 2022/0367033 A1
(43) Pub. Date: Nov. 17, 2022

(54) METHODS, SYSTEMS, AND DEVICES FOR A BIOPHILIA-BASED ECOSYSTEM TO PROMOTE WELLBEING AND PRODUCTIVITY USING MACHINE LEARNING

(71) Applicant: Humegy Corp., Raleigh, NC (US)

(72) Inventors: Claris Castillo SINGH, Raleigh, NC (US); Ray IDASZAK, Raleigh, NC (US)

(21) Appl. No.: 17/815,810

(22) Filed: Jul. 28, 2022

Related U.S. Application Data
(63) Continuation of application No. PCT/US21/15516, filed on Jan. 28, 2021.
(60) Provisional application No. 62/968,186, filed on Jan. 31, 2020.

(51) Int. Cl. G16H 20/70; G06N 5/02; G16H 40/67; A01G 7/04

(52) U.S. Cl. CPC G16H 20/70; G06N 5/022; G16H 40/67; A01G 7/045

(57) ABSTRACT

Systems and methods for increasing a user's task performance and personal wellbeing resulting from caring for indoor plants are disclosed. Internet-of-Things sensors and devices monitor measurements relating to environmental factors and a user's care patterns associated with designated indoor plants. User-performance sensors and devices measure the user's wellbeing and the user's task performance, both in caring for the plants and in other productivity-driven tasks. Recommendations for the plant care and prescriptions for the users are generated using machine-learning models and provided to the user through notifications to alter the user's behavior in a desirable way, both in plant care and in real-world task performance.

### CROSS-REFERENCE TO RELATED APPLICATIONS

This application is a bypass continuation of International Patent Application No. PCT/US21/15516 filed on Jan. 28, 2021, by Humegy Corp. entitled “METHODS, SYSTEMS, AND DEVICES FOR A BIOPHILIA-BASED ECOSYSTEM TO PROMOTE WELLBEING AND PRODUCTIVITY USING MACHINE LEARNING”, which claims priority to U.S. Provisional Patent Application No. 62/968,186 filed on Jan. 31, 2020, by Humegy Corp. entitled “METHODS, SYSTEMS, AND DEVICES FOR A BIOPHILIA-BASED PRODUCTIVITY AND WELLBEING SYSTEM USING MACHINE LEARNING,” the entire contents of all of which are incorporated by reference herein.

### TECHNICAL FIELD

The methods, systems, and devices disclosed herein relate to a biophilia-based ecosystem for increasing individual and/or team performance and wellbeing in a work or living environment using the beneficial effects of direct interaction with plants. More specifically, the methods, systems, and devices disclosed herein create beneficial user engagement and direct user interaction between users of the ecosystem (e.g., office workers and home residents) and plants that are or will be in the future located in their environment (e.g., office and home), which generates increased user productivity, performance, and wellbeing as well as increased plant health.

### BACKGROUND

Biophilia refers to the innate biological connection between humans and nature, and it is the basis of a rapidly growing trend in the interior space-design industry where a relationship is forged between science, the built environment, and nature, where nature is often in the form of indoor plants. There is an increasing body of scientific literature demonstrating the tangible measurable benefits of incorporating indoor plants into the built environment. The benefits stem in part from observed improved physiological markers such as heart rate, blood pressure and skin conductivity; and, range from improving patient recovery time in hospitals to increasing corporate employee productivity and wellbeing.

Caring for indoor plants is typically based on the proper recurring combination and application of water, nutrients, pesticides, and environmental factors such as sunlight, interior location, soil content and moisture, plant pot size, humidity, and temperature. A typical user determines the proper application and combination of each of these through trial-and-error, or by either reading articles written by or interacting with subject-matter experts ranging from professional plant suppliers to career botanists. All these approaches, variations and corresponding implementations may yield different results on the state of indoor plants, ranging from ill-looking to a healthy growing plant. Variations on this include individualized plant-care instructions accessible by a Quick Response (QR) code attached to a label on the plant prior to purchase where after purchase the user subsequently scans the QR code with their smartphone, taking them to a website with specific care instructions for that plant. More recently, electronic devices integrated into the plant pot directly and/or placed in the plant soil can read limited environmental data (such as sunlight, soil content and moisture, humidity, and/or temperature) and provide that data to the user in the form of smartphone or visual display reminders on the electronic device to attend to the needs of their plant.

However, these above-mentioned approaches to plant-care rely on the user's constant motivation and/or discipline. They also rely on the user being willing to care for the plant simply for the sake of caring for the plant, with no apparent additional benefits aside from keeping the plant alive.

But there are additional benefits of caring for plants. The improved physiological conditions and consequent improved productivity and wellbeing that come from having plants in a work or living space stem from their ability to keep an individual psychologically engaged through the sensory experience (visual aesthetics, texture, fragrance), sharing the plant caring experience with others, and the feelings of inner peace and the positive emotions that plants have been shown to elicit in people. Although, as there are increasing devices to assist users in the care of indoor plants, and as there is increasing literature and practice of the real scientific health and productivity benefits that indoor plants provide, current approaches lack the recognition of the benefit to the caregiver of engaging in caring for indoor plants and measuring and improving the performance of such activities; it is implicit or indirect at best. Instead, existing solutions focus on pseudo-automating plant-caring activities and/or rely on mobile phones, smart watches, or similar smart display as primary interface between the user and the plants. Behind this reality is the perception that gardening and plant caring activities are typically considered outdoor activities not suitable to living spaces, much less work environments. The implications of this trend are troubling, since modern society spends 90% of its time indoors and, according to the United Nations, three-fourths of the population will live in cities by 2050. Thus, the gap between humans and nature continues to widen.

Industry invests in productivity, healing rate, and learning rate improvements in the form of professional consulting to assess current performance and prescribe improvements based on changes in environment, culture, procedure, equipment, and/or finances including refined incentive and reward structures. As an example, insurance companies have recently begun working with industry to provide incentives as financial reward based on employees wearing Fitbit®-like wrist devices that measure steps and heart-rate embellished with reminders and summaries in achieving target goals associated with these measurements. This results in lower insurance rates for both the industry and individual by measured tasks such as increased steps walked per unit time resulting in healthier employees with fewer and less costly sick days taken.

By placing indoor plants around a person given a task, the rate at which that task is successfully achieved is measurably improved. However, even though electronic devices exist to measure the health of the plant, it is only implicit that the plant provide the aforementioned benefits without measuring the performance of the individual team or employee in the specific task of plant caregiving, nor does the machine intelligence exist to infer critical environmental factors, such as placement of plant relative to the location of windows or HVAC vents, the type of or specific plant best suited for a target environment, the interrelationship of these, and so forth.

Accordingly, there is a need for new methods, systems, and devices that facilitate plant-care and plant-selection activities in a way that measurably increases performance and wellbeing of people living and working in residential or office environments.

### SUMMARY

Disclosed herein are methods, systems, and devices for increasing individual and/or team wellbeing and performance in a work or living environment promoting the beneficial effects of direct interaction with plants. More specifically, methods, systems, and devices are disclosed that create beneficial user engagement and direct user interaction between users of the ecosystem (e.g., office workers, home residents) and plants selected for and located in their environment (e.g., office and home), which generates increased measurable productivity, performance, and wellbeing, as well as increased plant health.

The system disclosed herein solves the above-identified problems by providing an ecosystem where technology bring humans and nature closer by helping humans to keep plants alive and healthy while plants promote human's wellbeing by eliciting feelings of calmness and peace. At the heart of this ecosystem is novel technology that enhances the human-nature relationship by promoting their direct interaction centered on the human's wellbeing. The system combines the use of electronic measurements relating to the care and/or environmental factors of one or more indoor plants combined with measured performance of the plants' caregivers in caring for the plants to drive increased human task performance and wellbeing in real-world tasks, for example industry tasks, that are fundamental to that industry's primary operation and success. Measured and improved performance of individuals or teams caring for plants can be directly tied to driving improved performance in real-world corporate tasks, such as the coding of software, or other primary tasks of modern corporations for which productivity, healing rate, and/or learning rate improvements is beneficial.

The system disclosed herein further solves the above-identified problems by providing a platform that allows sharing and socialization of performance of a plant caregiver, which enables broad visibility to individual and team plant caregiver performance while also enabling greater accountability in plant caregiving.

The system disclosed herein further solves the above-identified problems by integrating contextual user performance information such as software bug fixes, heart rate, plant health, plant-care activities (e.g., time of watering), to gain insight into users' plant caregiving productivity and to prescribe ways to improve it while at the same time improving a target task performance of that user and their wellbeing.

The system disclosed herein further solves the above-identified problems by collecting and aggregating plant-health data and contextual data thereof to prescribe plant-care instructions that are precisely tailored to the needs of the specific plant and its environment.

The system disclosed herein further solves the above-identified problems by collecting and aggregating plant environmental data and contextual data thereof to prescribe specific plant or plant-type information in the selection and installation of a specific plant or plants for the methods, systems, and devices described herein.

The system disclosed herein further solves the above-identified problems by measuring and improving sensory engagement information between user and their plant, socialization of their plant, and responsiveness in administering plant caregiving, and contextual data thereof in calculating wellbeing attributes to prescribe specific ways to improve user wellbeing attributes.

The system disclosed herein further measures and improves performance of plant caregivers by collecting and combining various types of information, for example, the health of the plant, the health of the caregiver, and environmental factors (among others) available through third-party data sources to produce metrics and measurements that can be translated into high-level scores that capture the performance of the plant caregiver. The system disclosed herein positively correlates high scores with the caregiver's ability to anticipate (and address) the needs of the plant without being prompted or alerted.

The system disclosed herein is customizable and adaptive to conditions of the plant, caregiver, environment, and the like.

The system disclosed herein further solves the above-identified problems by providing incentives driven by measured performance (e.g., score) of the plant caregiver. For example, the incentives may, based on the scores, activate ambient elements, such as an air purifier or aromatic oil diffuser to reward the sensory experience and the perceived quality of the air and environment surrounding the plant caregiver. The incentives may also enable the logical grouping of plant caregivers and the mapping of the resulting group to a single plant to promote collaboration and engagement among team members or household members and contribute to the calculation of a wellbeing score (discussed in more detail below).

The system disclosed herein further solves the above-identified problems by providing the use of “natural interfaces” to enable communication between the user and plants. For example, natural interfaces that are soothing interfaces that blend with the natural themed surroundings in contrast to current solutions that rely on mobile apps as primary interface vehicle.

The above advantages are provided by a system in accordance with the principles of the embodiments described herein. The system includes Internet of Things (IoT) devices; external platforms; databases; data archiving, sharing and analysis; a processing unit for mapping incentives; and a set of incentive rules.

The system disclosed herein further solves the above-identified problems by providing an interface for providing messages to plant caregivers generated by the system. The messages may include, for example, status of environmental factors associated with the plants as collected continuously by the system platform from the IoT devices and stored in a data management system. Scores are continually computed by the system as a function of user response time, IoT device values, and completeness of task, i.e. how complete all the elements of the task have been performed. Higher scores are computed and assigned to the users if the users anticipate the needs of the plant without needing to be reminded by the system. The system collects all scores from all users and allows users to socialize them on their social networks, e.g. Facebook® or the like.

The system disclosed herein further solves the above-identified problems by using machine learning (ML) algorithms to compute combinations of values and actions by the user that constitute a healthy plant and support the performance and wellbeing of the caregiver, with the computations being based on data collected from all users, plants, third-party data sources, and contextual information thereof. For example, if a subset of 1,000 registered users of the system each are caregivers for an Anthurium indoor plant, then environmental values and caregiving patterns of the caregivers are continually collected by the system for those 1,000 Anthurium plants. Then, as any existing or new user within the system experiences a particular challenge with the health and/or caregiving for their Anthurium plant, the system may provide specific instructions as to the caregiver for how to best address and reconcile that particular challenge based solely on the application of ML algorithms of the collected data of what has already worked successfully for current caregivers of Anthurium plants. As an example, the system described herein may propose that a Monstera deliciosa is a better suited plant to be installed in place of a given Anturium plant similarly based on this application of ML algorithms of the collected data of what best works successfully for current caregivers of Anthurium plants. Advantageously, this system eliminates the need for the plant caregiver to consult professional plant suppliers, career botanists, or the like. Also advantageously, this system eliminates the need for any information from the caregivers as the system delivers the exclusively computationally derived optimal and specific caregiving instructions. These caregiving instructions, which may include instructed changes to environmental factors of the plant and/or changes in caregiving patterns, can be transacted within the system through a web interface or similar user interface on a PC or smartphone, augmented-reality glasses, virtual-reality displays, smart watches, or the like. An extension of this is collecting values from two different types of plants within the same environment and having the system ascertain what is beneficial or harmful in terms of interaction between the two, or more, plants.

The system disclosed herein further solves the above-identified problems by including incentives that are integrated into the system such that the system triggers them upon computing that certain thresholds of higher scores have been achieved by the users. For example, by incorporating ambient indoor enhancers such as air purifiers controlled by the ecosystem, the perceived quality of air can be improved, impacting the caregiver's wellbeing. This can be achieved by integrating IoT devices for these elements with the system such that they are triggered upon certain thresholds of higher scores being computed and achieved. Given a set of incentive rules, the system can compute a logical grouping of plant caregivers and map to these incentives. This facilitates collaboration and engagement where, for example, a team of plant caregivers is assigned to a single plant from the logical grouping of plant caregivers computed by the system.

A method implemented on at least one computing device for increasing user engagement and wellbeing using interaction with plants in a work environment is disclosed. The method includes receiving, at a back-end server, measurements representing plant-health data from a set of one or more IoT sensor devices associated with a plant. The method further includes receiving, at the back-end server, measurements representing user-performance data for a user from a set of one or more user-performance monitoring devices associated with the user. The method further includes generating a plant-care recommendation for the plant based on the measurements representing plant-health data, the plant-care recommendation being generated using a machine learning model. The method further includes generating a prescription with caregiving instructions for the plant based on the user-performance data. The method further includes calculating a CARE_SCORE value for the user. The CARE_SCORE value indicates an overall performance value for care pattern of the user. The method further includes transmitting a notification from the back-end server to a computing device of the user, the notification providing the plant-care recommendation and the prescription to the user.

In some embodiments of the method, the user-performance data include calculating a wellbeing score indicating a state of the user's wellbeing attributes.

In some embodiments of the method, the back-end server is implemented on a server within a cloud computing environment.

In some embodiments of the method, the IoT sensor devices include one or more of an air quality monitor, a plant-health monitor, a soil-moisture monitor, a temperature and humidity sensor, a gyroscopic plant rotation sensor, a light sensor, and a magnetometer electronic compass.

In some embodiments of the method, the one or more user-performance monitoring devices includes a wearable heartrate monitor, a wearable respiration monitor, and a wearable neural activity monitor.

In some embodiments of the method, the CARE_SCORE value is calculated based on a weighted calculation of received sensor data over a rolling time window.

In some embodiments the WELLBEING_SCORE value is calculated based on weighted number of user engagement with their plants, responsiveness in prescribed engagement with their plants, quantity of socialization of their plants on social media, timeliness of socialization of their plants on social media, and user-performance monitoring devices.

In some embodiments of the method, the notification is provided by emitting varying colors of light from an LED at varying intensity to represent the plant-care recommendation or the prescription.

In some embodiments of the method, the notification is provided by emitting sounds from a speaker of varying musical parameters such as overtone, timber, pitch, amplitude, duration, melody, harmony, rhythm, texture, structure, and temp to represent the plant-care recommendation or the prescription.

A server that provides a back-end of a system for increasing user engagement and wellbeing using interaction with plants in a work environment is disclosed. The server includes a memory. The server includes at least one processor. The processor is configured for receiving, at a back-end server, measurements representing plant-health data from a set of one or more IoT sensor devices associated with a plant. The processor is further configured for receiving, at the back-end server, measurements representing user-performance data for a user from a set of one or more user-performance monitoring devices associated with the user. The processor is further configured for generating a plant-care recommendation for the plant based on the measurements representing plant-health data, the plant-care recommendation being generated using a machine learning model. The processor is further configured for generating a prescription with caregiving instructions for the plant based on the user-performance data. The processor is further configured for calculating a CARE_SCORE value for the user, wherein the CARE_SCORE value indicates an overall performance value for care pattern of the user. The processor is further configured for calculating a better-fit plant or plant type in the location of the current IoT plant sensor devices. The processor is further configured for transmitting a notification from the back-end server to a computing device of the user, the notification providing the plant-care recommendation and the prescription to the user.

In some embodiments, the user-performance data includes calculating data indicating a state of the user's wellbeing attributes.

In some embodiments, the server is implemented within a cloud computing environment.

In some embodiments, the IoT sensor devices include one or more of an air quality monitor, a plant-health monitor, a soil-moisture monitor, a temperature and humidity sensor, a gyroscopic plant rotation sensor, a light sensor, and a magnetometer electronic compass.

In some embodiments, the one or more user-performance monitoring devices includes a wearable heartrate monitor.

In some embodiments, the CARE_SCORE value is calculated based on a weighted calculation of received sensor data over a rolling time window.

In some embodiments, the notification is provided by emitting varying colors of light from an LED at varying intensity to represent the plant-care recommendation or the prescription.

In some embodiments of the method, the notification is provided by emitting sounds from a speaker of varying musical parameters such as overtone, timber, pitch, amplitude, duration, melody, harmony, rhythm, texture, structure, and temp to represent the plant-care recommendation or the prescription.

A system for increasing user engagement and wellbeing using interaction with plants in an environment is disclosed. The system includes a client-side component. The client-side component includes an indoor monitor device. The client-side component further includes a plant-health device. The client-side component further includes a user personal-health device. The client-side component further includes one or more IoT devices. The IoT devices include an indoor enhancer element. The IoT devices include a plant-health display. The IoT devices include a user health monitor. The system further includes a third-party component. The third-party component includes a user performance-monitoring source. The third-party component includes a weather monitoring service component. The third-party component includes a social network component. The system further includes a server-side component. The server-side component includes a data exchange portal for receiving measurements from the client-side component, the IoT devices, and the third-party component over an API. The server-side component includes a recommender component for generating a plant-care recommendation using a machine learning model that was trained using historical plant-care activities. The server-side component includes a prescriber component for generating caregiving instructions for a plant based on the measurements. The server-side component includes an incentivizer component for calculating a CARE_SCORE value for a user. The CARE_SCORE value indicates an overall performance value for care pattern of the user. The server-side component includes a notification component for transmitting the plant-care recommendation and the caregiving instructions to the user.

A method of generating a recommendation of plant-care for a user is disclosed. The method includes receiving a raw data sample from a sensor associated with a plant. The method includes classifying the raw data sample received from the sensor. The classification is performed using a machine-learning model. The method includes determining, based on the classification of the raw data sample, whether a user's care pattern for the plant indicates that the user's care of the plant is problematic for health of the plant. The method includes identifying a key differentiating feature set based on the classification of the user's care pattern. The key differentiating feature set is identified by comparing the user's care pattern to a known good care pattern for plants of a type similar to the plant. The method includes issuing a recommendation to the user for plant-care of the plant.

In some embodiments of the method, the machine-learning model was generated using supervised learning to reach a desired level of accuracy.

A method of generating a recommendation of plant placement for a user is disclosed. The method includes receiving a raw data sample from a sensor associated with a plant. The method includes classifying the raw data sample received from the sensor. The classification is performed using a machine-learning model. The method includes determining, based on the classification of the raw data sample, an optimal location for the health of the plant. The method includes issuing a recommendation to the user for placement of the plant in the determined optimal location.

A method of generating a recommendation of a plant type for a user is disclosed, the method includes receiving a raw data sample from a sensor associated with a location. The method includes classifying the raw data sample received from the sensor. The classification is performed using a machine-learning model. The method includes determining, based on the classification of the raw data sample, an optimal plant type for the location. The method includes issuing a recommendation to the user for the determined optimal plant type.

A method of generating a recommendation of reward type for a user is disclosed, the method includes receiving historical user performance data sample that allows the biophilia-based ecosystem to analyze users' wellbeing, performance, and/or productivity. The method includes determining, based on the analysis of the raw data sample, an optimal reward type for the user. The method includes issuing a reward recommendation that improves the perceived conditions of the user's indoor space as well as provide assets of value to the user, e.g., digital badges, gift cards, tickets to sporting or other types of events, or the like.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

FIG. 1 depicts a high-level overview of an example of the biophilia-based ecosystem for promoting wellbeing and productivity.

FIGS. 2A and 2B depict an exemplary high-level architecture for implementing the high-level overview of the biophilia-based ecosystem shown in FIG. 1.

FIG. 3 depicts a block diagram illustrating components of a server that implements the back-end server of the biophilia-based ecosystem shown in FIGS. 2A and 2B.

FIG. 4 depicts an exemplary high-level flowchart for a process flow for generating a prescription for a user.

FIGS. 5A-5B depict exemplary high-level flowcharts describing a machine learning approach for training and using models to generate recommendations for a user.

FIGS. 6A-6C depict exemplary flowcharts for the CARE_SCORE algorithm.

FIG. 7A depicts an exemplary level-mapping function L(S_j\hat{ }i).

FIG. 7B depicts an exemplary level-mapping function L′(S_j\hat{ }i) for compass sensors.

FIG. 7C depicts an exemplary severity/importance function.

FIG. 7D depicts an exemplary responsiveness function.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Disclosed herein are systems, methods, and devices that use electronic measurements relating to the care and/or environmental factors one or more indoor plants combined with measured performance of the plants' caregivers in caring for the plants to drive increased human task performance and wellbeing in real-world tasks that are fundamental to that industry's or person's primary operation and success.

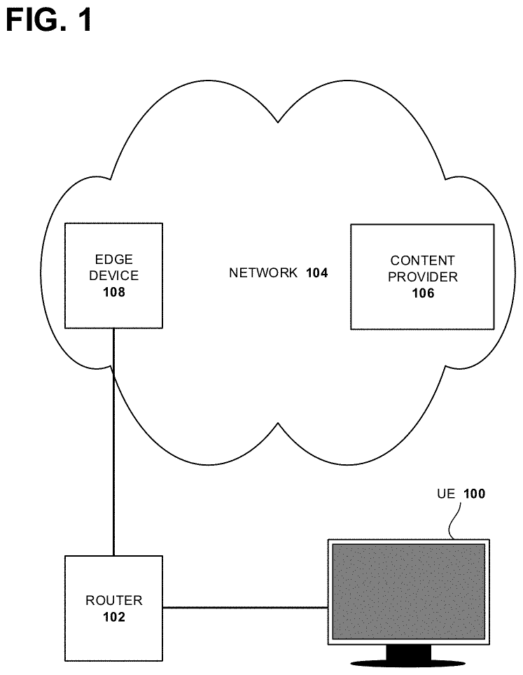

FIG. 1 depicts a high-level overview of an example of the biophilia-based ecosystem for promoting wellbeing and productivity.

Referring to FIG. 1, the biophilia-based ecosystem 100 shown in FIG. 1 uses plant-related information collected by sensors located on or near plants within an office environment, home environment, or other environment in combination with user-related performance information collected by sensors on or near users within the office environment, home environment, or other environment to generate and provide recommendations and incentives to the users to cause the users to better engage both with the plants in their surrounding as well as their tasks. As used herein, environment may include a corporate business office as well as a home environment.

The biophilia-based ecosystem 100 monitors the health of indoor plants, combines and analyzes the plant-health data with user-performance data and other contextual data including but not limited to weather, geographic information, indoor environment conditions, and data from social platforms to improve the performance and wellbeing of humans. The ecosystem 100 creates software models that represent plants, users, and indoor environments. The ecosystem 100 integrates these software models with the collected data and identifies novel relationships that can be used to improve human task performance and wellbeing via the adoption and care of plants. Based on the analysis of historical data, the ecosystem 100 prescribes (via a Prescriber component 106) and recommends (via a Recommender component 108) plant-care instructions that are tailored to the needs of the plants, the surrounding environment, the user, and other contextual information. To keep the user accountable and further support their needs, as well as the needs of the plants, the ecosystem analyzes historical data over time to incentivize (via an Incentivizer component 104) plant caregiving through a customizable rewards system that improves the perceived conditions of the user's indoor space as well as provide assets of value to the user, e.g., digital badges, gift cards, tickets to sporting or other types of events, or the like.

The biophilia-based ecosystem 100 receives plant-related data 110 that allows the system to measure users' performance of plant-care activities. As shown in FIG. 1, the ecosystem 100 receives user-related data 112 that allows the ecosystem 100 to measure users' 114 wellbeing, performance, and/or productivity. The plant-related data 110 and user-related data 112 is received by a back-end system 102 of the biophilia-based ecosystem 100. As shown in FIG. 1, the back-end system 102 includes an Incentivizer component 104, a Prescriber component 106, and a Recommender component 108. These components work together to provide notifications 116 to the user 114. The notifications 116 prompt the user 114 to engage with either the plants 118, their tasks 120, nature, a specific individual (e.g., a coworker), or the like. These components further generate and provide rewards information 122. When the user 114 successfully engages with either the plants 118 or their tasks 120, or both, the user 114 earns the rewards 122, creating a positive feedback loop that leads to better plant health, better user wellbeing, and better user performance.

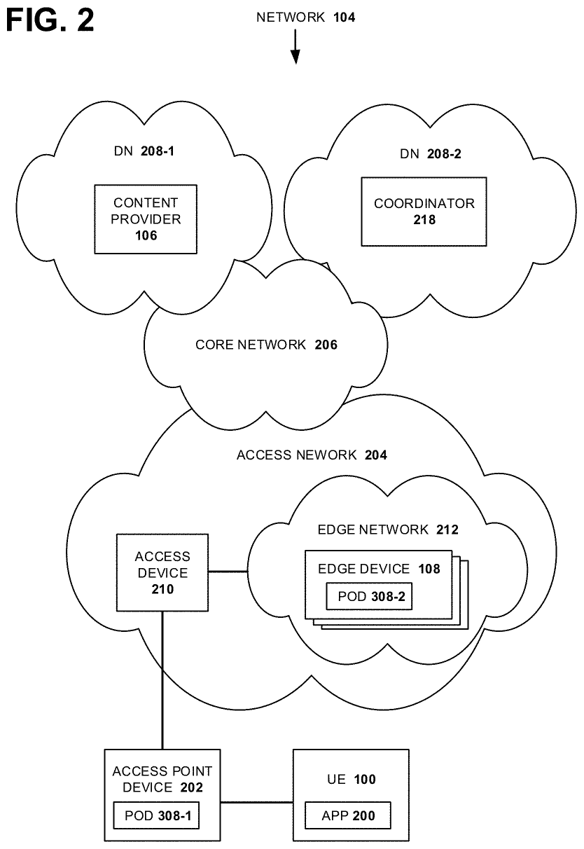

FIGS. 2A and 2B depicts an exemplary high-level architecture for implementing the high-level overview of the biophilia-based ecosystem shown in FIG. 1.

Referring to FIGS. 2A and 2B, the biophilia-based ecosystem can be divided into a back-end server 202, one or more front-end devices 204 that communicate over a network 206 with the back-end server 202, and one or more third-party platforms 208 that communicate over a network 206 with the back-end server 202 and/or the one or more front-end devices 204. The back-end server 202 includes a Server-Side Component (SSC) 210. The one or more front-end devices 204 are part of a Client-Side Component (CSC) 212. The one or more third-party platforms 208 are part of either a Third-Party Component (TPC) 214 (as shown in FIG. 2) or a Third-Party Client-Side Component (TCC) (not shown in FIG. 2).

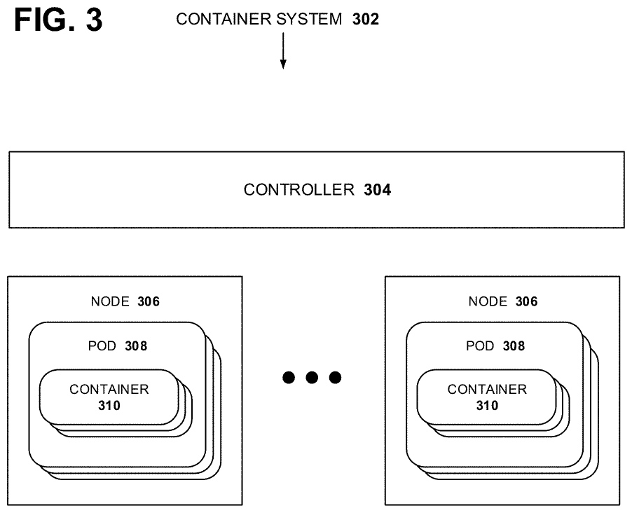

FIG. 3 depicts a block diagram illustrating components of a server that implements the back-end server of the biophilia-based ecosystem shown in FIGS. 2A and 2B.

The server 300 is configured to host a least some of the components that make up the biophilia-based ecosystem described herein. The server 300 may include at least one of a processor 302, a main memory 304, a database 306, a data exchange portal 308, and a communication interface 310. The server 300 may be configured to host a virtual server. In some embodiments, the virtual server may be distributed over a plurality of hardware servers (such as server 300).

The processor 302 may be a multi-core server class processor suitable for hardware virtualization. The processor may support at least a 64-bit architecture and a single instruction multiple data (SIMD) instruction set.

The main memory 304 may include a combination of volatile memory (e.g. random-access memory) and non-volatile memory (e.g. flash memory). The database 306 may include one or more hard drives. The database 306 may provide at least a portion of the functionality of the data archiving function of FIG. 2.

The data exchange portal 308 may provide one or more high-speed communication ports to datacenter switches, routers, and/or network storage appliances. The data exchange portal 308 may include high-speed optical Ethernet, InfiniBand (IB), Internet Small Computer System Interface iSCSI, and/or Fibre Channel interfaces. The data exchange portal 308 may further include one or more application programming interfaces (APIs) for facilitating data exchange with the devices and/or platforms that make up the biophilia-based ecosystem.

The communication interface 310 may provide an administrative user interface (UI) that supports local and/or remote configuration of the server 300 by an administrator. The communication interface 310 may further provide a user portal that allows users to access the biophilia-based ecosystem, either through a web-based portal/dashboard or through a mobile application (e.g., smartphone application).

Referring back to FIG. 2, the CSC 212 comprises a set of one or more IoT devices 216 that can be further classified into plant-health monitoring devices 218 (e.g., soil moisture sensor), user wellbeing monitoring devices 220, user performance monitoring devices 222, and/or personal health monitoring devices 224 (e.g., Fitbit®, user respiration monitors, and the like), indoor-space monitoring devices 226 (e.g., temperature, humidity, air quality monitor), indoor environment enhancers 228 (e.g., aromatic oil diffusers), display devices 230 (e.g., plant-health display devices). The one or more IoT devices 216 are embedded in plant pots or into the plants or soil, or located in the environment near the plants being monitored.

The TPC 214 comprises one or more third-party social platforms 232, such as Facebook® and Instagram®, that provide contextual information about the user and enables the socialization of activities supported by the ecosystem. The SSC 210 may integrate or connect with the third-party social platforms 232 of the TPC 214 through standard APIs 234.

The TCC comprises public and/or private third-party data sources that provide information relevant to the biophilia-based productivity system. For example, the TCC includes weather-monitoring services 236, such as the Weather Channel, which provide information relevant to the geolocation of the plant and can be used to gain insight into the conditions of the indoor environment surrounding the plant. The TCC further includes performance-monitoring sources 222 and health-monitoring sources 224, such as Fitbit monitoring records, a company's employee performance records, or software development bug-tracking software, for example.

The SSC 210 comprises a software system that is accessible over the Internet to communicate with the CSC 212, the TPC 214, and the TCC mentioned above, as well as support the methods and subsystems described herein.

The SSC 210 may be implemented as a back-end system, for example, as shown in FIG. 2. The SSC 210 may be hosted in a cloud-computing environment and may be implemented on one or more servers, as shown in FIG. 3. The SSC 210 includes a non-transitory computer-readable medium that includes a plurality of machine-readable instructions which when executed by one or more processors of the one or more servers are adapted to cause the one or more servers to perform the methods of the biophilia-based ecosystem described herein to improve plant health in the system as well as increasing user engagement and user wellbeing and performance of the users caring for the plants.

For example, the SSC 210 performs a method for increasing user engagement and wellbeing by encouraging optimal interaction with plants in an environment. The method includes receiving, at the SSC 10, plant-care measurements that are transmitted over a network from a set of one or more Internet-of-Things (IoT) sensor devices. As explained herein, the plant-care measurements represent plant-health data. The method further includes receiving, at the SSC 210, user-performance measurements that are transmitted over a network from a set of one or more user-performance monitoring devices associated with a user. As explained herein, the user-performance measurements represent user-performance data for the user. The method includes generating, using a machine-learning model implemented at the SSC 210, a plant-care recommendation based on the plant-care measurements representing plant-health data. The method further includes calculating, based on the received plant-care measurements and the received user-performance measurements, a CARE_SCORE value for the user. As explained in more detail herein, the CARE_SCORE value indicates an overall performance value for a care pattern of the user. The method further includes generating a prescription having caregiving instructions for the user based on the user-performance data. The method further includes transmitting, from the SSC 210, a notification to a computing device associated with the user.

The SSC 210 may be implemented on a virtual (i.e. software-implemented) server in the cloud-computing environment. The virtual server may be implemented as a separate operating system (OS) running on one or more physical (i.e. hardware-implemented) servers. Any applicable virtual server may by be used. The virtual server may be implemented within the Microsoft Azure®, Amazon Web Services (AWS®), Google Cloud Platform (GCP®), or the like. In other embodiments, the SSC may be implemented on one or more servers in a networked computing environment located with a business facility and/or another datacenter.

The SSC 210 may be configured to communicate over the Internet, over low-power long-range wide-area wireless IoT network (LoRa), and/or over narrowband IoT (NB-IoT) with the CSC 212, the TPC 214, and/or the TCC. The components may be configured to communicate over at least one of an hypertext transfer protocol (HTTP) session, an HTTP secure (HTTPS) session, a secure sockets layer (SSL) protocol session, a transport layer security (TLS) protocol session, a datagram transport layer security (DTLS) protocol session, a file transfer protocol (FTP) session, a user datagram protocol (UDP), a transmission control protocol (TCP), a lightweight messaging protocol (MQTT), and a remote direct memory access (RDMA) transfer. The communication between the components may use a standard interface, for example, and application programming interface (API).

As shown in FIG. 2, the SSC 210 may include a data-sharing function 238, a data-archiving function 240, a data-analysis function 242, a data exchange/portal function 244, and a notification function. The data-sharing function 238 provides integration with social networks, such as Facebook®, Instagram®, and the like. The data-archiving function 240 provides storage for system data (e.g., a database). The data-analysis function 242 provides user-performance scoring 248 and determines user-performance improvement prescription 246 using ML algorithms. The SSC 210 may further include an Incentivizer component 104 (as discussed in the context of FIG. 1), a Recommender component 108 (as discussed in the context of FIG. 1), and a Prescriber component 106 (as discussed in the context of FIG. 1).

The CSC 212 receives input from various sensor devices to collect plant health and related information such as soil moisture, sunlight exposure, orientation (compass direction), geographic location (altitude, latitude, and longitude), air humidity, among others. The CSC 212 communicates with the SSC 210 over the Internet, LoRa, and/or NB-IoT (i.e., network 206) using standard communication protocols, where the SSC 210 provides further aggregation and analysis of the received data. The CSC 212 may comprise, for example, an Arduino or Raspberry Pi processor connected to one or more of various types of low-cost sensors that are intelligently placed with the plant to collect measurements related to its health conditions, e.g., soil moisture and air humidity.

The CSC 212 may be configured to group devices into clusters based on various criteria, including, for example, their physical proximity, common needs of the plants they support, convenience, or other criteria. Clusters may be identified manually or automatically. For automatic clustering, the methods may rely on short distance communication protocols, for example, Bluetooth®, and powered by the data analysis system to determine commonality of plant needs. These clusters of plants may share sensors and be co-managed by the biophilia-based ecosystem based on their common needs, environmental conditions, or other factors.

The SSC 210 aggregates, analyze, and archives plant health data, user data, environment data and contextual information collected by elements of CSC 212 for the purpose of improving human task performance. The ecosystem may include an IoT platform that streams in data from IoT devices using light-weight communication protocols like MQTT and has built-in data analytic framework to support offline and real-time analysis and extensive data management operations such as storage, sharing, archival of the sensor data. Additionally, the system may also preferably support more robust communication protocols to transact with social platforms, e.g., Facebook® and weather services, e.g., Weather Channel®, also built into the CSC 212, for broader data integration and analysis of contextual information.

The SSC 210 creates software logical representations of users, plants and environment parameters and related information to facilitate their software manipulation. For example, the SSC 210 may create a data model for plants, users, and indoor factors that is implemented into an unstructured database for efficient access, linkage, and analysis, and can also be accessed using the standard interfaces of the system.

The SSC 210 determines and prescribes, based on analysis of information collected from the data sources in the TPC 214 and the CSC 212, plant-care instructions tailored to the specific needs of the plant, the user, and the indoor environment. The SSC 210 then communicates the prescribed plant-care instructions to the user for their execution. The plant-care instructions may be communicated in a number of ways, as described in more detail below.

The SSC 210 prescribes plant-care instructions (via Prescriber component 106) based on analysis of aggregated historical plant-health data, plant-care activities, user performance data, and environmental data associated with the indoor space and geographic location of instances of the same plant. The SSC 210 may trigger a notification 250 indicating to the user that a plant may need to be relocated or misted with water due to an observable decline of air humidity in the early months of winter, which may result from an overly dry indoor space or close proximity between a plant and a vent. On the other hand, the SSC 210 may not falsely or incorrectly determine that a plant that is receiving low light exposure on an extremely cloudy day or week should be relocated. Such an avoidance of false positives or false triggers is advantageous over existing plant-care IoT devices, which provide feedback on the immediate state of a plant, rather than precision plant healthcare through meaningful, comprehensive analysis of historical plant health-related data.

The user may be notified of prescribed instructions, for example watering the plant, rotating the plant, relocating the plant, adding grow lights emulating beneficial sunlight frequencies when relocating the plant is not feasible, fertilizing the plant, or the like. Such notifications may be provided to the user by the plant health device 218 of the CSC 212. In one embodiment, the plant health device 218 may emit faint lights following a predefined color scheme to indicate specific plant caring instructions. For example, a blue glowing light may indicate to water the plant, and a yellow glowing light may indicate to relocate the plant to a location with more natural light. In one embodiment, the notifications may be provided using advanced augmented reality (AR) capabilities integrated and triggered via a mobile application. For example, when viewing the actual plant through a camera of a mobile device (e.g., smartphone), the mobile application may overlay the prescribed instructions and/or the glowing light scheme on the plant to easily indicate to the user how to implement the prescribed plant-care instructions.

The SSC 210 may take into account user performance information obtained from consumer personal IoT health devices 224 including but not limited to heart rate and breathing rate to prompt and notify the user with plant-care instructions when the user is in better disposition to execute the instructions or when the plant-care activity itself has the potential to improve the user's performance or wellbeing by, for example, facilitating a calming effect on the user. In one embodiment, the SSC 210 may prompt the user to care for a plant, thereby alluring the user's attention to nature, when the user's personal health device 224 indicates that the user shows signs of sustained stress for an extended period of time (e.g., during a particularly stressful period of work).

The biophilia-based ecosystem allows a user to perform authentication to claim a particular plant-care activity, such as watering at the time of the activity or to retrieve information related to the user, plants under the user's care, or any other information available in the SSC 210 to which the user has access. The authentication may be implemented using standard authentication mechanisms, such as password-base via web interface against the health station, or other more indirect approaches such as scanning of a QR code on the surface of the plant pot from a mobile app where the user is already authenticated, Bluetooth Rx/Tx also built into the plant health device, etc. User authentication for performing a task may be used, for example, in cases where multiple care givers are responsible for a single plant.

The SSC 210 includes a Recommender component 108 for providing placement recommendations for a plant. The Recommender component 108 infers, based on data obtained from CSC 212 devices including a compass (orientation) and sunlight sensor (light exposure); weather service (e.g., sunrise and sunset time); and time of day, the placement of a plant relative to near-by sunlight sources (e.g., windows). The orientation of the compass and the sunlight sensor may be strategically aligned to determine the orientation of light sources, such as windows. For example, a plant receiving light on a sensor facing east from around sunrise time until around noon indicates that the plant is facing an east window. This information may be combined with light-intensity measurements by the same light sensor to provide insight into the proximity of the plant to the window. This information may be correlated with information from other plants in the same geographical region for fine tuning plant-care instructions.

The placement recommendations for a plant provided by the Recommender component 108 may include recommending a particular plant or plants that would thrive in one or more specific locations based on data collected from sensors in or near those specific locations. Those sensors may already be installed in an existing plant at those locations, or they may be put at those specific locations separately (e.g., without an associated plant) to gather environmental data from the locations being considered to allow the Recommender component 108 to recommend the best type of plant and/or best location for future addition.

The biophilia-based ecosystem's analysis of this data may trigger a recommendation notification to the user indicating that the plant should be relocated to meet the light requirements of the plant. In one embodiment, the system leverages information about plants collocated in the same office or building and potentially under the same ownership to recommend swapping locations to better align the need of the plants with the available sunlight in the building. In another embodiment, the system leverages information about plants collocated in the same office or building and potentially under the same ownership to recommend a new different specific plant or plant type be placed in the location of the current plant sensor because it will perform better there.

The biophilia-based ecosystem's analysis of this data may trigger a recommendation notification to the user identifying a set of plants that would thrive in a given environment based on the conditions observed in it. An extended version of this method may recommend pleasantly aesthetic arrangements of these plants including additional accessories such as tables, hangers, or others.

The Prescriber component 106 may prescribe the rotation of the plant pot to ensure the uniform exposure of the plant leaves to sun light. In such a scenario, the orientation of the sunlight sensor is aligned with the north of the compass, and the plant's orientation is tracked over time. The Prescriber 106 may user sunlight exposure data to infer when a plant does or does not need to be rotated due to uniform light exposure in the surrounding space where the plant is placed. For example, the Prescriber 106 may prescribe rotating the plant in 45-degree increments weekly.

The SCC 201 can infer the location of a window relative to a plant. even when is not explicitly known by the system, by gathering sunlight exposure information via one or more light sensors in the pot and combining this information with weather services information, e.g., sunrise and sunset times, and information from the magnetometer and the compass.

The SSC 210 may infer estimated GPS coordinates of a plant using data provided by a GPS system built into other devices collocated with the plant, for example, a plant caregiver's mobile device. In one embodiment, the SSC 210 may receive a scanned QR code from a mobile application on a user's smartphone or mobile device that is scanned by the user off of a QR code placed on the surface of the plant pot. The mobile application may read the mobile device's GPS location using an API at the time the QR code is scanned, and transmit that GPS location to the SSC 210, where the plant's location information in the SSC's data model may be automatically updated. To verify the plant's stored GPS location information when a plant gets moved to a different location, the SSC 210 may prompt the user to update GPS location periodically by prompting the user to re-scan the QR code.

The historical and derived data stored in the SSC 210 may be accessed and/or retrieved through web API interfaces 234 that can be accessible through modern mobile, augmented-, mixed-, or virtual-extended-reality devices or any other authorized third-party software. In one embodiment, a user may access a plant's health statistics by scanning a QR code on the plant's pot using, for example, the user's smartphone or mobile device. For example, a user's mobile application may be used to scan the QR code and display an avatar narrating the health statistics of the plant, the performance information of the user, or any other pertinent information. Similarly, the data may be accessed using a dashboard via a web interface that provides deep insight and statistics of the history of the plant and other contextual information regarding user and indoor environment.

The SSC 210 may compute a performance score of a plant caregiver in performing the task of caring for a plant with the purpose of rewarding users that anticipate the needs of a plant before being prompted by the prescriber. For example, a user who anticipates watering a plant before the soil moisture reaches a certain predefined threshold will receive a higher score compared to a user who waters a plant immediately after the prescriber prompts the user to water the plant.

The Prescriber component 106 and Recommender component 108 may be further configured to optimize prescription and recommendation planning to ease the plant-caring activities on behalf of the user. For example, when multiple plants require water within a short time window, e.g., 1 day, all watering prescriptions may be issued at or for the same time to reduce the number of individual watering activities for the user.

The Prescriber component 106 and Recommender component 108 may further be configured to identify plants whose needs are met by the conditions of a given environment while incorporating user's preferences such as types of plants, size of plants, cost, maintenance, etc. and other constraints imposed by the environment and recommend pleasantly aesthetic arrangements of these plants to the user. Such arrangements may include accessories such as tables and hangers which can be made accessible for purchase through the platform. Augmented-, mixed-, virtual-, or extended-reality displays may superimpose the plants' arrangements over an image of the designated space provided a priori by the user.

As explained, the biophilia-based ecosystem described herein aggregates sensor data from multiple plants and users to determine users' plant-caring and behavioral patterns, and then to prescribe actions that lead to changes in those behavior patterns and/or the adoption of new behavior patterns. Sources of data that may be used to infer plant caring and behavioral patterns include but are not limited to calendar, wearable IoT devices, social media interactions, etc. For example, the system can optimize the notification schedule to maximize the possibilities that all users caring for a given plant are made aware of the notification, all while observing the plant's health. In this context, the communication medium of the notification could be a natural interface such as a lighting pattern on the pot, a sound emitted by a device connected to the system, a push notification on the phone, etc. For example, the system could infer that one user is only at the office on Fridays and delay any notification to care for the plant prompted on Thursday to Friday to ensure that the user has the opportunity to benefit from interacting with plants through the system.

Additionally, the biophilia-based ecosystem described herein may recommend a set of plants that meet the environment conditions observed while optimizing for other objectives, such as, for example, user preferences, native plants, leafy plants, variety, quantity and cost, etc.

The biophilia-based ecosystem described herein may create logical (and physical) communities, e.g., family, for caring for plants where plant caring activities can be shared, delegated, promoted and rewarded.

The biophilia-based ecosystem described herein may recommend and track, for their wellbeing score, socialization of user interactions with their plant, e.g. scores, rewards, on a social media platform, e.g. Facebook® or the like.

The SSC 210 allows an administrator associated with the account of one or more users to assign one incentive model from a set of predefined and customizable incentive models to each user. Examples of incentive models include: (1) External Incentives: issuing of an item of value such as a pass to a self-improvement class; (2) Better Workspace Incentives: activation of aromatic oil diffusers located in the office space for a predefined time window to improve the sensory experience of the caregiver and the quality of the perceived indoor ambience; and (3) Team Building Incentives: issuing of an item/event of value to the whole team such as a bowling party or team lunch.

The Incentivizer component 104 may input a user score into an incentive model that issues rewards selected by an administrator responsible for the account associated with the user. The Incentivizer component 104 may issue rewards to users that achieve some measure of success such as, for example, keeping a score above certain value (threshold) more than 75% times in the last 30 days.

The Team Building Incentives model 254 allows for the logical grouping of plants and users as well as assigning multiple caregivers to a plant for supporting social group activities such as team building and sharing of household chores. For example, the Team Building Incentives model may assign multiple caregivers with various roles to a plant where all parties are located in the same office space to promote accountability and communication between group members. To reward teams or team members with high scores, the Incentivizer may issue rewards that further promote team building such as social events.

The Better Workspace Incentives model 256 rewards users by activating IoT devices that improve the perceived conditions of their indoor environment for a predefined period of time. For example, such IoT devices may include, for example, aromatic oil diffusers, air purifiers, sophisticated indoor lighting systems, or the like. The indoor enhancer IoT devices are part of the CSC 212 and are integrated and in communication with the SSC for transacting data and instructions.

The SSC 210 combines data from external user-performance monitoring frameworks with plant-care activities including score, plant-health data, etc. for the purpose of prescribing ways of improving a user's performance at performing certain task. For example, the SSC may integrate information from software code development bug-tracking software, calendar, emails, and plant-care activities to prompt a software engineer to care for their plant in order to pull their attention to the plant when software statistics suggest a decline in performance and/or productivity or a rush period of software development.

The SSC 210 may mine user activity patterns, plant-health data, indoor and outdoor environmental factors, time, and other contextual information to prompt a feedback request from the plant caregiver on their perceived wellbeing when caring for plants. In one embodiment, the SSC 210 may favor times of the day when the plant is under the sunlight to enhance feelings of connectedness to nature or times of the day when data obtained from user-performance devices indicate that the user may have a better disposition to provide such feedback.

The feedback provided by the plant caregiver, either unprompted or in response to the feedback request, may be provided through mechanisms such as a mobile device associated with the user (e.g., smartphone or smart watch), through the user's dashboard, or through input mechanisms located at or near the plant. Such user-provided feedback may be used to further the intelligence of the system in understanding the ecosystem of plants and caregivers, optimize its operation and performance and extend its capabilities to integrate with other third-party systems such as social networks, or the like. For example, the feedback mechanism may consist of sharing a photo of the plant, labeling the plant with a health status (e.g., “well and healthy”), a literal message, a rank, etc. Images shared by users with the system may be provided as input to the machine learning to identify patterns of plant health, caregiver wellbeing score, and any other contextual information thereof.

The biophilia-based ecosystem described herein promotes mindfulness, that is, bringing the user's full attention into the contemplation of and caring for a plant, by preferably monitoring the breathing rate of the user while the contemplation and care-giving activity takes place. For example, the system may integrate a CSC 212 device on a plant pot with a user's device (e.g., a user's performance device or a user's mobile device) such that the device on the plant pot may be activated to measure the breathing rate of the user when the user, having the user device on or about them, comes within a close distance of the plant pot. Similarly, respiration may be measured directly via radio high-frequency transmitted to, reflected from, and received by the plant pot component and time-of-flight reflected and measured directly from dimensional changes in the user's torso as they breathe.

The biophilia-based ecosystem described herein promotes mindfulness, that is, bringing the user's full attention into the contemplation of and caring for a plant, by monitoring the neural activity of the user while the contemplation and care-giving activity takes place. For example, the system may integrate a CSC 212 neural activity wearable device, e.g., Neuralink®, Neurosity®, that measures the users' neural activity during contemplation of their plant or plant caregiving such that colored LED lights incorporated within the pot emit different hues corresponding to different neural activity. For example, agitated neural activity may emit red lighting on the user's plant pot whereas relaxed neural activity may emit blue lighting.

The biophilia-based ecosystem facilitates partnership of precision plant care where a purchaser of a system IoT device gifts the IoT device with a plant to a recipient, and where both the purchaser and recipient, as the key actors, have user accounts with the system and the recipient is designated the primary plant caregiver. The platform may then be configured to inform both the purchaser and caregiver of precision plant health care-giving needs of a single shared plant, in effect forming a partnership where the purchaser and caregiver may or may not be co-located. For example, the gifted plant may be configured with plant grow lights beneficial to plant growth, and these grow lights configured to be operable on or off by the system. Upon receiving notification from the system that the gifted plant could benefit from activation of the grow lights on a recurring schedule, at times the purchaser in different geographic region than the recipient and plant may initiate an action to trigger the grow lights on instead of the plant recipient. This triggering may be initiated, for example, by a mindfulness activity preferably monitored (e.g., breathing rate) by an IoT device in the purchaser's proximity such that the greater the purchaser's monitored mindfulness, the more complete the required grow light dose to the plant in the recipient's geographic location. The IoT device monitoring the purchaser's mindfulness may be attached to a plant in the purchaser's proximity such that it can be used to contribute beneficial grow light dosing to either the purchaser's or the recipient's plant (and vice-versa), and CARE_SCORE and WELLBEING_SCORE scores for both (described in more detail below) computed by the system. The system facilitates these partnerships such that system-connected IoT devices may be used to contribute to the caregiving of a different individual's plant in any combination of one-to-one, one-to-many, many-to-one, and many-to-many configurations. The system can also modularize the system elements of the partnership to be applicable, for example, to a corporate environment where the key actors are supervisor and direct report, or the like. In this case, the supervisor may provide a thank you to the direct report that in whole, or in part, is associated with a particular LED display or the like on the IoT device associated with the direct report's plant, denoting that special recognition bestowed upon them by their supervisor. Similarly, the direct report may acknowledge their manager with a thank you through the system via a particular LED display on the manager's IoT device associated with their plant.

The biophilia-based ecosystem may be configured to offer integration capabilities to allow third-party services/devices (TPC, TCC) to provide personalized resources to caregivers and plants based on data captured by the system. Examples of standard methods for integration with third-party providers are open APIs and publish-subscribe communication mechanisms. These methods enable third-party service/supply providers to offer their services in response to caregiver needs captured by the biophilia-based productivity system. For example, through standard publish-subscribe mechanisms, a supplier of misting bottles may subscribe to messages published by the system about caregivers having reported issues relating to dry environments. The misting-bottle supplier may, based on this information, issue recommendations in the form of an advertisement or tip displayed on the user's dashboard or mobile app, or in the form of a free misting bottle in exchange of a review or any other thing of value. Aspects addressed by recommendations are not limited to plant but also cover the caregiver and the environment.

The biophilia-based ecosystem disclosed herein allows for integration between the system and corporate and/or other organization performance and productivity systems by enabling the exchange of values between these systems to improve individual and/or organization performance and productivity. For example, a corporate or other organization system may determine that the performance or productivity of an employee is falling as measured, for example, by the number of bugs increasing in lines of software code at a gaming software company or mistakes made in administering healthcare in a hospital. In such a situation, the system instructs and/or provides an incentive to the identified lagging employee to provide caregiving, as described herein, to a plant with one or more connected IoT devices. The resulting reduction in stress and increase in concentration and attention is correlated and applied concomitantly to both the plant and employee's monitored care-giving resulting from the instruction from the system disclosed herein, and the organization's goal for that employee in realizing a positive and directly measurable increase in employee performance and productivity via integration of the two systems.

Components of the Server-Side Component (SSC)

The Incentivizer component 104, Prescriber component 106, and Recommender component 108 of the SSC 210 are described here in more detail.

The biophilia-based ecosystem collects observations from IoT devices intelligently placed to accurately monitor the health of the plant, third-party network-accessible devices and any other source of information that can be used to gain insight into the health of the plant and its relationship with the plant's caregiver. The sampling rate of the data collection process may be configurable and dynamically learned by the system based on historical information collected about the plant, the caregiver and other contextual information thereof, such as geolocation and the like. Sensor data from the IoT devices may be fed into the components directly or indirectly through post-processing methods that prepare the data for processing as needed by downstream components.

The Prescriber Component

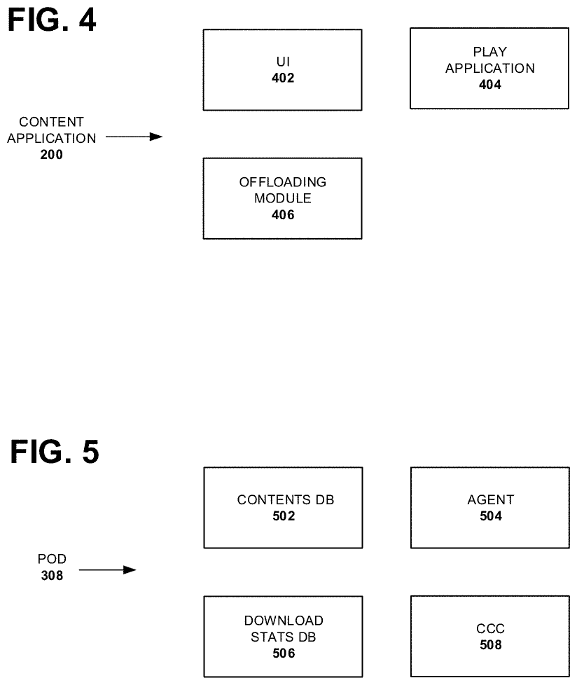

FIG. 4 depicts an exemplary high-level flowchart for a process flow for generating a prescription for a user.

The Prescriber component 106 analyzes data collected from the CSC 212, the TPC 214, and other data sources to identify conditions in the plant that require attention from the caregiver. Based on the identified conditions, the Prescriber component 106 may issue a notification to the user. The notification may include, in addition to information about the plant's health, instructions (i.e., a prescription) for how the user can address the identified issues. The Prescriber component 106 may use information about the caregiver in determining instructions for the plant-care prescription, such as the caregiver's stress levels and/or work activity. By using such information, the Prescriber component 106 aims at improving the effectiveness of the notification in supporting the caregiver by engaging the caregiver into an activity that improves the caregiver's work performance while addressing the needs of the plant in a timely manner. The algorithm of the Prescriber component 106 is extensible and independent of the notification mechanism used and can be modified to work with any notification mechanism without sacrificing generality.

To measure the health of the plant, the Prescriber component 106 quantifies properties of the plant, for example, soil moisture, relative to the known quantified needs of the plant. The needs of the plant are preferably known in advance, either based on specifications of the plant type or through machine learning using historical information collected from the multiple plants and caregivers in the system. A notification may be a function of two parameters: severity and responsiveness. Both of these parameters may be used by the notification system to increase the effectiveness of the notification in accurately reflecting the state of the plant and prompting action from the caregiver to address it. Severity refers to how serious is the status of the property in consideration, e.g., the soil moisture sensor measures 0. Responsiveness refers to the caregiver's level of responsiveness to the condition in consideration. If this condition has been impoverishing amid multiple notifications issued to the caregiver, such information is reflected by the notification system. For example, in an exemplary system using LEDs to issue notifications, the color of the LED may indicate the sensor type, the brightness may indicate the severity of the condition, and the frequency at which the LED changes intensity may indicate the responsiveness.

In some embodiments, the notifications may be configured to interact with a location-tracking device associated with a user, such as the user's mobile phone, or smart watch. In such embodiments, the notifications may be provided to the user when the user is near the plant to which the notification applies, as determined by comparing the user's location based on the location-tracking device, and the known location of the plant to which the notification applies. Thus, for example, where the notification system is colored LED lights on the plants, the lights may be triggered when it is determined that the user is near the plant. Similarly, where the notification system is a notification to the user's device, such as a mobile device or smart watch, the notification may be triggered to appear on the user's device when it is determined that the user is near the plant.

As shown in FIG. 4, the Prescriber component 106 checks for received sensor data, at step 402. The sensor data is represented as S_j\hat{ }i, where S represents a particular sensor measurement, i represents the sensor type of the sensor, and j represents particular instance of the sensor measurement. For example, S_3\hat{ }4 represents the third instance of a measurement from a sensor type 4, which may be, for example, a compass. The value of S_3\hat{ }4 represents the value of that particular instance of the sensor measurement. If sensor data has been received, then the Prescriber component 106 moves on to step 404, where it performs pre-processing of the received sensor data. After performing pre-processing of the received sensor data, the Prescriber component 106 calculates a level based on the received sensor data, at step 406. The level is calculated using a step function to match the sensor measurements to a particular level, as shown in step 408. The calculated level indicates the severity of the plant health with respect to the type of sensor for which the data was received. For example, if the sensor data is received from the soil moisture sensor, then the level calculated at step 406 using the step function in step 408 represents the soil moisture of the plant. If the calculated level is normal (as determined at step 410), then the notification parameters are reset at step 412, and the Prescriber component 106 continues to check for received sensor data, back at step 402. If the calculated level is not normal, then the Prescriber component 106 calculates a severity based on the calculated level and/or the received sensor data, at step 414. The severity is calculated using an importance mapping function that maps each level to a numerical value between 0 and 1, at step 416, to be used as a weight in the algorithm for computing a CARE_SCORE (discussed in more detail below). After the severity has been calculated at step 414, the Prescriber component 106 calculates responsiveness, at step 418, using a responsiveness function that degrades proportionally to the time it takes for the user to act on the notification issued for the sensor type, as shown in step 420. After the responsiveness has been calculated, the Prescriber component 106 issues a notification for a prescribed activity to the user, at step 424. The prescribed activity is based on the caregiver's personal and performance information, at step 422, such that the prescribed activity has the best chance of being implemented by the user, leading to beneficial results for the plant, and/or leading to beneficial results for the user's performance or productivity.

The Recommender Component

The Recommender component 108 handles aspects of the biophilia-based ecosystem that relate to aspects of health of the plants that can be improved but do not hinder their sustainability and survival. Recommendations determined by the Recommender component 108 may be translated into a prescription recipe. Similarly, prescriptions determined by the Prescriber component 106 may be translated into recommendations. To produce recommendations, the Recommender component 108 uses algorithms to determine beneficial courses of action for particular plants or groups of plants. The Recommender component 108 uses machine learning models using some or all types of data collected within the ecosystem or from outside the ecosystem. The data used by the Recommender component 108 includes but is not limited to plant-health data, caregiver information, and contextual information. For example, the Recommender component 108 may recommend reducing the watering frequency for a plant by comparing the caregiver's watering pattern to the watering pattern of thousands of other caregivers in the system with the same plant type who have provided explicit positive feedback about their plant, such as, a photo, a numerical or other rating, notes, or the like.

The Recommender component 108 may recommend that a new different plant or plant type should be placed in the location of the current sensor due to the calculation that it will perform best in that location if there is not a current plant there (e.g., sensor only with no plant), or better than the current plant with sensor in that location.

The Recommender component 108 may recommend socialization of their plants on social media. Data is collected by the system on the quantity of social media posts and responsiveness of the user since the time to post in following the recommendation.

FIGS. 5A and 5B depict exemplary high-level flowcharts describing a machine learning approach for training and using models to generate recommendations for a user. The flowcharts shown in FIGS. 5A and 5B may be implemented in the Recommender component 108.

The Recommender component 108 classifies good and/or bad care patterns for a given plant by mapping explicit feedback, e.g., “plant is very healthy,” into labels that indicate how good or bad the caregiving pattern of the caregiver user is. As new caregivers join the ecosystem, the algorithm compares the new users' caregiving patterns to existing caregiving patterns and results and issues recommendations that are likely to yield a healthier plant.

For example, the Recommender component 108 may use information about plants physically co-located within the same building and possibly under the same owner, windows orientation and proximity and their sunlight exposure patterns to recommend actions such as swapping location of plants to better fit their light needs to the lighting available in the office building.

Referring to FIG. 5A, the training process begins with raw data samples from the sensors in the system, at step 502. The raw data samples are labeled, at step 504, using explicit feedback on plant health received from the user. After the data is labeled, supervised learning is performed to train a neural network (e.g., using back propagation to generate or train a neural network), at step 506. A check is performed to see if a desired level of accuracy has been reached with the supervised learning, at step 510. If the desired level of accuracy has not been reached, the process starts over with another raw data sample, at step 502. If the desired level of accuracy has been reached, then the machine learning model is set, at step 512.

Referring to FIG. 5B, once the machine learning model has been set, the machine learning model is used to generate recommendations for the user. At step 552, raw data samples for a particular plant, designated as plant k, are received. At step 554, the raw data samples are fed into the set machine learning model to classify the care pattern based on the raw data samples. At step 556, it is determined if the classified care pattern is troubling. If the classified care pattern is not determined to be troubling, then the process exits and waits to receive new raw data samples, at step 552. If the classified care pattern is determined to be troubling, then a key differentiating feature set is identified for the care pattern, at step 558, by comparing the classified care pattern against existing (e.g., known) good care patterns for plants similar to the plant being analyzed, as shown in step 560. Based on the identified key differentiating feature set, one or more recommendations are issued to the user, at step 562.

The Incentivizer Component

The Incentivizer component 104 handles generating and issuing rewards to caregivers who exhibit good performance in caring for their plants by preferably meeting goals established either by themselves or others with administrative roles. Rewards issued by the Incentivizer component 104 may be fully customizable and redeemable externally or through any component in the system, including the CSC, TPC and SSC. To measure performance, the Incentivizer component 104 quantifies the performance of the caregiver by analyzing data about the health of the plant, the caregiver, the environment and other contextual information. In one embodiment, the Incentivizer component 104 produces a measurable performance value, which may be referred to, for example as a CARE_SCORE. In one embodiment, the Incentivizer component 104 produces a value that indicates the overall health state of the plant based on the health factors the user tends to and/or are provided by the environment.

The PLANT_HEALTH_SCORE Calculation

The PLANT_HEALTH_SCORE is determined for each plant following a suitable time period, which may be either time-based or event driven. The ecosystem disclosed herein organizes data samples for each sensor into a rolling time window of pre-defined length and processes the samples on a period-of-time basis. Samples within a time window are processed together to produce a score for each sensor type, referred to as the SENSOR_T which is the distance between the average of all samples and the optimal or desired value for that specific parameter and sensor. SENSOR_T is a measure of the overall health of the plant based on the parameters being monitored by the sensors and may be used as input to other processes in the system.

The WELLBEING_SCORE Calculation

The WELLBEING_SCORE is determined for each caregiver following a suitable time period, which may be either time-based or event driven. The WELLBEING_SCORE is calculated to be an attribute correlating the caregiver's CARE_SCORE with data based on personal health device, weighted number of caregiver engagements with their plants, responsiveness in prescribed engagement with their plants, quantity of socialization of their plants on social media, timeliness of socialization of their plants on social media, and user-performance monitoring devices. The prescriber component may initiate a prescription to initiate an interaction between the caregiver and plants for the purpose of alleviating observable low values of the WELLBEING_SCORE.

The CARE_SCORE Calculation

The CARE_SCORE is determined for each caregiver following a suitable time period, which may be either time-based or event driven. The ecosystem disclosed herein organizes data samples for each sensor into a rolling time window of pre-defined length and processes the samples on a period-of-time basis. Samples within a time window are processed together to produce a score for each sensor type, which may be referred to, for example, as a SENSOR_SCORE, and an overall score, which may be referred to, for example, as a CARE_SCORE, which is a weighted sum of the SENSOR_SCORE values for all sensor types. The weight associated with each sensor is configurable and dynamically updated to reflect the needs of the plant, the user and the environment surrounding the plant. The length of the time window may be proactively learned using machine learning.

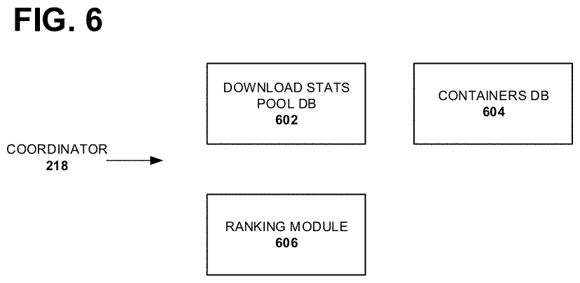

FIGS. 6A-6C depict exemplary flowcharts for calculating the CARE_SCORE.

Referring to FIG. 6A, it is determined, at step 602, whether all sensors have been considered. If all sensors have been considered, then the determined CARE_SCORE is reported, at step 604. If all sensors have not been considered, then at step 606, it is determined whether all samples in the rolling time window have been considered. The samples in the rolling time window may be represented, for example, as S\hat{ }i [1, . . . , N]. If all the samples in the rolling time window have been considered, then the process flow moves to the process flow shown in FIG. 6C. If all the samples have not been considered, then at step 608, it is determined whether weather information has been used for the sensor measurements. If weather information has not been used for the sensor measurements, then checking for whether all samples have been considered continues, at step 606. If weather information has been used for the sensor measurements, then it is determined, at step 610, if the sensor measurements come from a compass sensor. If the sensor measurements are not from a compass sensor, then an importance weight value is calculated at step 612 using the importance mapping function described in in more detail below, as shown in step 614. After the importance weight value has been calculated at step 612, the process flow moves to the process flow shown in FIG. 6B. If the sensor measurements are from a compass or gyroscopic sensor, then it is determined whether the rotation has reached the minimum threshold value, at step 616. If the rotation has not reached the minimum threshold value, then an importance weight value is calculated at step 620 using the compass-specific importance mapping function described in more detail below, as shown in step 622, and the process flow then moves to the process flow shown in FIG. 6B. If the rotation has reached the minimum threshold value as determined at step 616, then the time of the last sample step is updated at step 618 and the process flow then moves to the process flow shown in FIG. 6B. The time of the last sample is updated in step 618 according to the equation T_last (S\hat{ }i)=T(S_j\hat{ }i).

FIG. 6B depicts a portion of the exemplary process flow for calculating the CARE_SCORE. The equations used to calculate the CARE_SCORE are shown in FIG. 6B using the variables and functions described below. When the process flow shown in FIG. 6B is completed, the algorithm returns to the process flow shown in FIG. 6A to determine whether all samples in the rolling time window have been considered.

FIG. 6C depicts a portion of the exemplary process flow for calculating the CARE_SCORE. The equations used to calculate the CARE_SCORE are shown in FIG. 6C using the variables and functions described below. When the process flow shown in FIG. 6C is completed, the algorithm returns to the process flow shown in FIG. 6A to determine whether all sensors have been considered.

The variables and functions used to calculate the CARE_SCORE value, as well as descriptions of the variables and functions, are provided in more detail below.

S_j\hat{ }i refers to a particular sensor measurement instance j for sensor type i. The multiple sensor type for each sensor type i may be represented as i=1, . . . , M. This representation shows that there are M sensor types in the system.

The rolling window of time is represented as S_j\hat{ }i [1, . . . , N]. The rolling window of time includes samples or observations S from sensor type i consisting of N samples. One epoch, as referred to herein, consists of one rolling window of time.

The score of a sample, referred to as SAMPLE_SCORE(S_j\hat{ }i), is calculated by factoring in the importance value (I) of the value sampled, the responsiveness (R) of the user, and the temporality or age (T) of the sample in the relationship to the epoch or rolling window of time, using the following relationship: SAMPLE_SCORE(S_j\hat{ }i)=T_w (S_j\hat{ }i) R_w (S_j\hat{ }i)·I_w (S_j\hat{ }i).

The score for a sensor of sensor type i, referred to as SENSOR_SCORE(S\hat{ }i), is calculated over the SAMPLE_SCORE(S_j\hat{ }i) for all the samples in the rolling time window as follows:

Σⱼ₌₁^{N}SAMPLE_SCORE(S_j\hat{ }i)

The normalized SENSOR_SCORE(S\hat{ }i) value is shown as follows:

\overline{SENSOR_{SCORE(S\hat{ }i)}}.

The CARE_SCORE represents the overall score of a user's plant-care pattern. The CARE_SCORE is calculated using the following formula:

Σᵢ^{M}W(S\hat{ }i)·\sqrt{SENSOR_SCORE(S\hat{ }i)}

The W(S\hat{ }i) represents the weight indicating importance of sensor S\hat{ }i, as compared to the other M−1 sensors. This is a configurable parameter that can be dynamically learned based on needs of the plant, user input, user patterns, environment, etc.

The time of the first sample in the rolling time window is represented as t_1. The time of the last sample in the rolling time window is represented as t_N. The time that has elapsed since the last time the user cared for the plant associated with S\hat{ }i, is represented as T_Care\hat{ }i.

The minimum number of degrees that a plant must be rotated regularly to receive sunlight uniformly is represented as Th_ROT. The maximum tolerable time that a plant can go without care for a given sensor type i is represented as Th_RES.

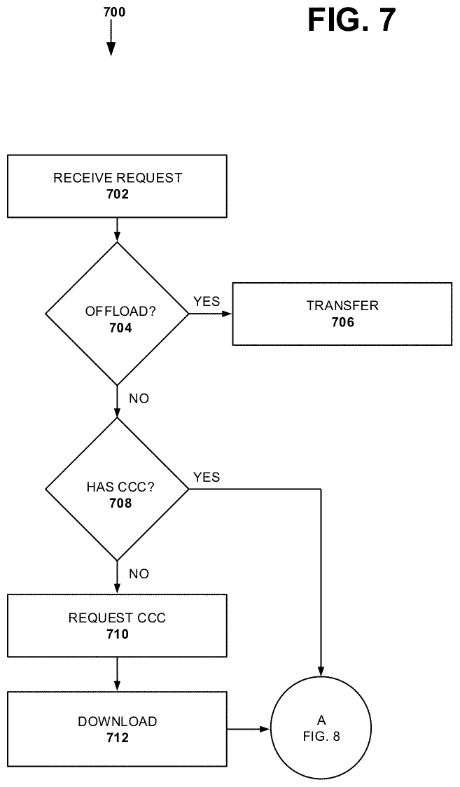

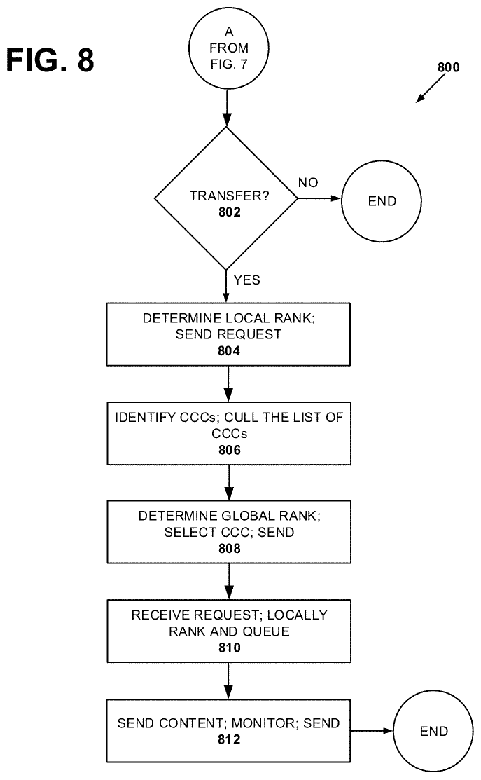

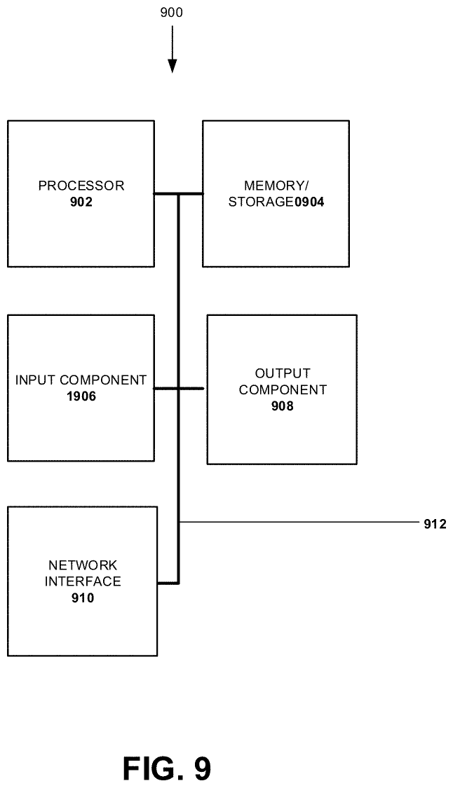

For each sensor type i, a level-mapping function L(S_j\hat{ }i) is defined that maps the sensor measurements to n levels (L). In one embodiment, the level-mapping function is a step function. The level indicates the severity of the plant health regarding one particular sensor type, e.g., soil moisture. In this function the y-axis consists of the level; and the x-axis corresponds to the j^{th} sample (S_j\hat{ }i). FIG. 7A depicts an exemplary level-mapping function L(S_j\hat{ }i).

For a compass sensor, the level-mapping function differs slightly from the level-mapping function for the other types of sensors. The level-mapping function L′(S_j\hat{ }i) represents the level-mapping function for compass sensors. In L′(S_j\hat{ }i), the x-axis corresponds to T_Care\hat{ }i. FIG. 7B depicts an exemplary level-mapping function L′(S_j\hat{ }i) for compass sensors. As can be seen in FIG. 7B, the longer amount of time since the plant was rotated, the worse the severity of the condition of the plant. Conversely, the shorter amount of time since the plant was rotated, the better the severity of the condition of the plant.

The completion of each epoch may trigger the issuing of a notification to the user and/or update of the user's CARE_SCORE. Such a notification is represented as N\hat{ }(S_j\hat{ }i)(I, R). In one embodiment, the notification may include two parameters: severity/importance (I), and responsiveness (R). Severity refers to the importance that the magnitude observed in the value of the sample (S_j\hat{ }i) has on the health of the plant. Responsiveness indicates the time elapsed between the last time the user was notified about this sensor creating a condition that requires action, e.g., watering, and the current and latest sample. Different notifications put in effect these parameters differently depending on its capabilities. For example, when using a fainted light emitter (LED), the color of the LED indicates the sensor type, the brightness of the light may indicate the importance (I) of the notification and the frequency at which the LED blinks may indicate the responsiveness (R). A mobile app could use the same or similar colors, but the user-interface design of a mobile app may present it differently given the app's richer display capabilities, as compared to an LED.

The severity/importance (I) is a non-decreasing function that maps each level to a numerical value between 0 and 1 to be used as a weight in calculating the CARE_SCORE and other values. The severity/importance function is represented as I(L(S_j\hat{ }i)) or I_w (S_j\hat{ }i). FIG. 7C depicts an exemplary severity/importance function. For example, in the case of an LED, the higher the severity of the parameter of the health of the plant being measured, the brighter the light emitted by the notification LED.

The responsiveness (R) is a function that degrades proportionally to the time it takes for the user to act onto the notification issued for sensor type i. It is a function of two parameters num and Δ (delta). Num refers to the number of sample intervals (epochs) since the last time a notification had been sent to the user with respect to sensor type i. Delta indicates the rate at which the function degrades. FIG. 7D depicts an exemplary responsiveness function. The responsiveness function is represented as R\hat{ }(S\hat{ }j) (num, Δ) or R_w (S\hat{ }i). In the case where an LED is used as notification mechanism, R is the frequency at which the light emitted by the LED blinks to notify the user of the latest sample and the condition of the plant.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. Programming languages may include, but are not limited to: JavaScript, Java, Python, Ruby, PHP, C, C++, C#, Objective-C, Go, Scala, Swift, Kotlin, OCaml, or the like. The program code may execute entirely on the user's computer, partly on the user's computer, as a stand-alone software package, partly on the user's computer, and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1-49. (canceled)

50. A method for increasing user engagement and wellbeing by encouraging optimal interaction with plants in an environment, the method comprising:
receiving, at a back-end server, plant-care measurements that are transmitted over a network from a set of one or more Internet-of-Things (IoT) sensor devices, wherein the plant-care measurements represent plant-health data;
receiving, at the back-end server, user-performance measurements that are transmitted over a network from a set of one or more user-performance monitoring devices associated with a user, wherein the user-performance measurements represent user-performance data for the user;
generating, using a machine-learning model implemented at the back-end server, a plant-care recommendation based on the plant-care measurements representing plant-health data;
calculating, based on the received plant-care measurements and the received user-performance measurements, a CARE_SCORE value for the user, wherein the CARE_SCORE value indicates an overall performance value for a care pattern of the user;
calculating a PLANT_HEALTH_SCORE for the plant, wherein the PLANT_HEALTH_SCORE indicates the overall health of the plant;
calculating a WELLBEING_SCORE value for the user, wherein the WELLBEING_SCORE value indicates the wellbeing attributes of the user; and
generating a prescription having caregiving instructions for the user based on the user-performance data; and
transmitting, from the back-end server, a notification to a computing device associated with the user, wherein the notification includes at least one of the plant-care recommendation for the plant and the prescription for the user.

51. The method of claim 50, wherein the plant-care recommendation includes at least one of the following: a recommended course of action to care for a plant associated with at least one of the set of IoT sensor devices, or a recommended location to place a plant of a specified type.

52. The method of claim 50, wherein the plant-care recommendation for the plant is further based on feedback from the user that indicates a health condition of the plant.

53. The method of claim 50, wherein the IoT sensor devices include one or more devices selected from the set of: an air quality monitor, a compass, an orientation sensor, a soil-moisture monitor, and a light sensor.

54. The method of claim 50, wherein the one or more user-performance monitoring devices includes a smart watch, a wearable device, a wearable heartrate monitor, a wearable respiration monitor, or a wearable neural activity monitor.

55. The method of claim 50, wherein:
the CARE_SCORE value is calculated based on a weighted calculation of received plant-care measurements from the IoT sensor devices received over a rolling time window,
the PLANT_HEALTH_SCORE value is calculated based on organizing data samples received from each IoT sensor device into a rolling time window and processing the data samples on a period-of-time basis, or
the WELLBEING_SCORE value is calculated to be an attribute correlating the CARE_SCORE with data based on personal health device, weighted number of caregiver engagements with their plants, responsiveness in prescribed engagement with their plants, quantity of socialization of their plants on social media, timeliness of socialization of their plants on social media, and user-performance monitoring devices.

56. The method of claim 50, wherein notification is transmitted to the computing device associated with the user upon a determination that the user is in proximity to a plant to which the plant-care recommendation applies.

57. The method of claim 50, wherein the notification includes at least one of the following:
emitting varying colors of light from an LED at varying intensity to represent the plant-care recommendation or the prescription, and
emitting sounds from a speaker of varying musical parameters such as overtone, timber, pitch, amplitude, duration, melody, harmony, rhythm, texture, structure, and temp to represent the plant-care recommendation, a severity level of the plant-care recommendation, or the prescription.

58. A server that provides a back-end of a system for increasing user engagement and wellbeing using interaction with plants in an environment, the server comprising:
a memory; and
at least one processor configured to:
receive, at the back-end, plant-care measurements that are transmitted over a network from a set of one or more Internet-of-Things (IoT) sensor devices, wherein the plant-care measurements represent plant-health data;
receive, at the back-end, user-performance measurements that are transmitted over a network from a set of one or more user-performance monitoring devices associated with a user, wherein the user-performance measurements represent user-performance data for the user;
generate, using a machine-learning model implemented at the back-end, a plant-care recommendation based on the plant-care measurements representing plant-health data;
calculate, based on the received plant-care measurements and the received user-performance measurements, a CARE_SCORE value for the user, wherein the CARE_SCORE value indicates an overall performance value for a care pattern of the user;
calculate PLANT_HEALTH_SCORE for the plant, wherein the PLANT_HEALTH_SCORE indicates the overall health of the plant;
calculate a WELLBEING_SCORE value for the user, wherein the WELLBEING_SCORE value indicates the wellbeing attributes of the user;
generate a prescription having caregiving instructions for the user based on the user-performance data; and
transmit, from the back-end server, a notification to a computing device associated with the user, wherein the notification includes at least one of the plant-care recommendation for the plant and the prescription for the user.

59. The server of claim 58, wherein the plant-care recommendation includes at least one of the following: a recommended course of action to care for a plant associated with at least one of the set of IoT sensor devices, or a recommended location to place a plant of a specified type.

60. The server of claim 58, wherein the plant-care recommendation for the plant is further based on feedback from the user that indicates a health condition of the plant.

61. The server of claim 58, wherein the IoT sensor devices include one or more devices selected from the set of: an air quality monitor, a compass, an orientation sensor, a soil-moisture monitor, and a light sensor.

62. The server of claim 58, wherein the one or more user-performance monitoring devices includes a smart watch, a wearable device, a wearable heartrate monitor, a wearable respiration monitor, or a wearable neural activity monitor.

63. The server of claim 58, wherein:
the CARE_SCORE value is calculated based on a weighted calculation of received plant-care measurements from the IoT sensor devices received over a rolling time window,
the PLANT_HEALTH_SCORE value is calculated based on organizing data samples for each IoT sensor device into a rolling time window and processing the samples on a period-of-time basis, or
the WELLBEING_SCORE value is calculated to be an attribute correlating the CARE_SCORE with data based on personal health device, weighted number of caregiver engagements with their plants, responsiveness in prescribed engagement with their plants, quantity of socialization of their plants on social media, timeliness of socialization of their plants on social media, and user-performance monitoring devices.

64. The server of claim 58, wherein the notification is transmitted to the computing device associated with the user upon a determination that the user is in proximity to a plant to which the plant-care recommendation applies.

65. The server of claim 58, wherein the notification includes at least one of the following:
emitting varying colors of light from an LED at varying intensity to represent the plant-care recommendation or the prescription, and
emitting sounds from a speaker of varying musical parameters such as overtone, timber, pitch, amplitude, duration, melody, harmony, rhythm, texture, structure, and temp to represent the plant-care recommendation, a severity level of the plant-care recommendation, or the prescription.

66. A method of generating a recommendation of plant-care for a user, the method comprising:
receiving a raw data sample from a sensor associated with a plant or a location;
classifying the raw data sample received from the sensor, wherein the classification is performed using a machine-learning model;
determining, based on the classification of the raw data sample, information about plant-care for the plant; and
issuing a recommendation to the user for plant-care of the plant.

67. The method of claim 66, further comprising identifying a key differentiating feature set based on the classification of the information about plant-care for the plant,
wherein the information about plant-care for the plant includes the user's care pattern for the plant,
wherein the user's care pattern for the plant indicates that the user's care of the plant is problematic for health of the plant, and
wherein the key differentiating feature set is identified by comparing the user's care pattern to a known good care pattern for plants of a type similar to the plant.

68. The method of claim 66, wherein the information about plant-care for the plant includes an optimal location for the health of the plant, and wherein the recommendation includes the determined optimal location.

69. The method of claim 66, wherein the information about plant-care for the plant includes an optimal plant type for the location, and wherein the recommendation includes the determined optimal plant type.
